(19) United States
(12) Patent Application Publication
(10) Pub. No.: US 2022/0257582 A1
(43) Pub. Date: Aug. 18, 2022

(54) TREATMENT FOR SYNUCLEINOPATHIES

(71) Applicant: SUN PHARMA ADVANCED RESEARCH COMPANY LIMITED, Mumbai, Maharashtra (IN)

(72) Inventors: Nitin Krishnaji DAMLE, Mumbai, Maharashtra (IN); Andrew Michael GOLDFINE, Plainsboro, NJ (US); Sanjaykumar Nandlal MANDHANE, Vadodara (IN)

(73) Assignee: SUN PHARMA ADVANCED RESEARCH COMPANY LIMITED, Mumbai, Maharashtra (IN)

(21) Appl. No.: 17/618,230

(22) PCT Filed: Jun. 9, 2020
(86) PCT No.: PCT/IB2020/055425

§ 371 Date: Dec. 10, 2021

(30) Foreign Application Priority Data
Jun. 11, 2019 (IN) 201921023164

(51) Int. Cl. A61K 31/47; A61P 25/28

(52) U.S. Cl. CPC A61K 31/47; A61P 25/28

(57) ABSTRACT

A method of treating or preventing synucleinopathies in a human subject comprising administering a therapeutically effective amount of a compound of Formula 1,

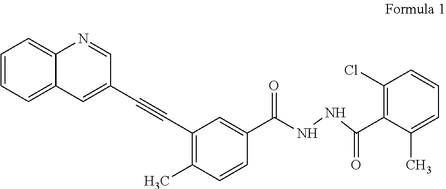 [SMILES: Cc1ccc(C(=O)NNC(=O)c2c(C)cccc2Cl)cc1C#Cc1cnc2ccccc2c1]

or its pharmaceutically acceptable salt.

### FIELD OF THE INVENTION

The invention relates to a method of treating or preventing synucleinopathies in a human subject comprising administering a compound of Formula 1

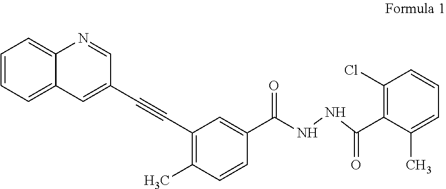 [SMILES: Cc1ccc(C(=O)NNC(=O)c2c(C)cccc2Cl)cc1C#Cc1cnc2ccccc2c1]

or a pharmaceutically acceptable salt thereof.

### BACKGROUND OF THE INVENTION

A number of neurodegenerative diseases are characterized by the accumulation of distinct misfolded protein inclusions. Development of such inclusions often initiates a series of biochemical events that eventually culminate into the programmed death of the affected neurons. One such protein is alpha synuclein (aSYN). Alpha synuclein (aSYN) is a member of a family of soluble proteins that includes alpha, beta and gamma synucleins. All synucleins have a common highly conserved lipid binding domain using which they associate with various phospholipid vesicles. Significant emphasis has been placed on mutations in alpha synuclein because these mutant alpha synucleins can cause autosomal, dominant, early onset, familial Parkinson's Disease (PD).

In addition to PD, there are at least two other distinct neurodegenerative conditions in which alpha synuclein has been implicated as a causative agent, and these are collectively referred to as synucleinopathies. Synucleinopathies are neurodegenerative diseases characterized by the abnormal accumulation of aggregates of alpha synuclein protein in neurons, nerve fibers, astrocytes or glial cells. There are three main types of central nervous system synucleinopathies: Parkinson's Disease (PD), Dementia with Lewy Bodies (DLB), also known as Lewy Body Dementia (LBD), and Multiple System Atrophy (MSA). In addition to these three major synucleinopathies, there is evidence that many patients with REM-sleep behavior disorder will go on to develop a synucleinopathy (McCann H et al., Parkinsonism & Related Disorders. 20 Suppl 1: S62-7, 2014). There are also rare disorders, such as various neuroaxonal dystrophies and primary autonomic failure, characterized by central or peripheral α-synuclein-based pathologies. (Kahle P J et al, Acta Neuropathol. 115(1):87-95, 2008; Goedert M et al., J Parkinsons Dis. 7 (s1): S53-S71, 2017; Lindholm D et al., Front. Aging Neurosci. 26; 8:254. 2016).

One of the key events associated with various neurodegenerative conditions including synucleinopathies is the increased expression and activation of a non-receptor protein tyrosine kinase, c-Abl. Once activated, c-Abl phosphorylates a diverse group of proteins (substrates of c-Abl) often altering their normal physiological functions. Alpha synuclein is one such substrate which is phosphorylated by c-Abl on Tyrosine 39. Once phosphorylated, alpha synuclein tends to form aggregates resulting in the formation of tendrils and fibrils that eventually go on to form Lewy Bodies that are often observed in the autopsied brains of patients with synucleinopathies such as PD and LBD. Evidence from genetic models of synucleinopathies in the CNS suggests that pre-formed fibrils of alpha synuclein are unable to cause neurodegeneration in the absence of functional c-Abl (Ko H S, et al., Proc Natl Acad Sci USA. 21; 107(38):16691-6, 2010). Given the dependence of alpha synuclein-initiated neurodegenerative processes on functional c-Abl, pharmacological inhibition using a small molecule inhibitor of c-Abl may provide therapeutically meaningful and advantageous neuroprotective effects.

PCT publication No. WO2012098416A1 discloses many specific compounds which have tyrosine kinase inhibitory properties. One of the compounds disclosed in the ‘416’ application is

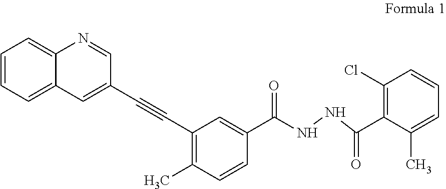 [SMILES: Cc1ccc(C(=O)NNC(=O)c2c(C)cccc2Cl)cc1C#Cc1cnc2ccccc2c1]

WIPO publication No. WO2017208267A1 discloses the use of the compound of Formula I for the treatment of Parkinson's Disease (PD).

Following references describe methods for treating neurodegenerative diseases using tyrosine kinase inhibitors:

US patent application No. US20150087653A1, US20170216287, US20140045826, US20060128720, US20050043264; U.S. Pat. Nos. 9,474,753, 7,910,586, 8,618,063B2 and WIPO application No. WO2012139027A1.

Although efforts to develop an effective treatment for PD and synucleinopathies has increased in past few years but effective disease-modifying alternatives and better symptomatic relief treatments are still necessary. Treatments or therapies developed for PD may or may not be effective for treating synucleinopathies like DLB and MSA. A person having ordinary skill in the art will have to carry out specific tests and trials to demonstrate the effectiveness of the therapy on these diseases.

### SUMMARY OF THE INVENTION

The present invention provides method of treating or preventing synucleinopathies in a human subject comprising administering a therapeutically effective amount of a compound of Formula 1,

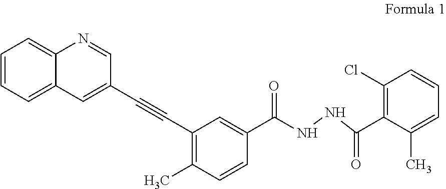 [SMILES: Cc1ccc(C(=O)NNC(=O)c2c(C)cccc2Cl)cc1C#Cc1cnc2ccccc2c1]

or its pharmaceutically acceptable salt thereof.

In another aspect, the present invention provides a method of treating or preventing synucleinopathies in a human subject comprising administering compound of Formula 1, or its pharmaceutically acceptable salt at a dose ranging from 0.1 mg to 1000 mg per day.

In another aspect, the present invention provides a method of treating or preventing synucleinopathies of the CNS, other than Parkinson's disease, in a human subject comprising administering compound of Formula 1,

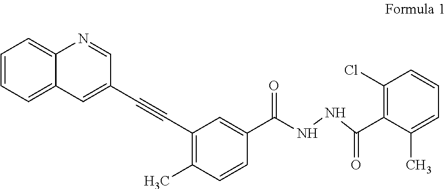 [SMILES: Cc1ccc(C(=O)NNC(=O)c2c(C)cccc2Cl)cc1C#Cc1cnc2ccccc2c1]

or its pharmaceutically acceptable salt at a dose ranging from 0.1 mg to 1000 mg per day.

### DESCRIPTION OF THE FIGURES

FIG. 1: Nigral TH^{+ve} cell counts (operated side).

FIG. 2: Striatal TH OD (operated vs non-operated sides).

FIG. 3: Striatal dopamine levels: Absolute values, operated vs non-operated sides.

### DETAILED DESCRIPTION OF THE INVENTION

In one aspect, the present invention provides a method of treating or preventing synucleinopathies in a human subject comprising administering a therapeutically effective amount of the compound of Formula 1,

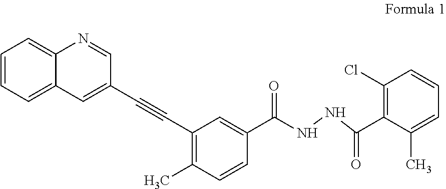 [SMILES: Cc1ccc(C(=O)NNC(=O)c2c(C)cccc2Cl)cc1C#Cc1cnc2ccccc2c1]

or its pharmaceutically acceptable salt thereof.

In another aspect the present invention provides a method of treating or preventing synucleinopathies in a human subject comprising administering compound of Formula 1,

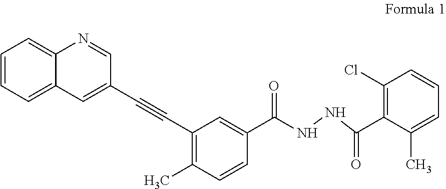 [SMILES: Cc1ccc(C(=O)NNC(=O)c2c(C)cccc2Cl)cc1C#Cc1cnc2ccccc2c1]

or its pharmaceutically acceptable salt thereof at a dose ranging from 0.1 mg to 1000 mg per day.

In an embodiment, the present invention provides a method of treating synucleinopathies in a human subject comprising administering the compound of Formula I or pharmaceutically acceptable salt thereof at a dose ranging from 10 to 500 mg per day. In an embodiment, the present invention provides a method of treating synucleinopathies in a human subject comprising administering the compound of Formula I or pharmaceutically acceptable salt thereof at a dose ranging from 100 to 600 mg per day, preferably the dose is 200 mg to 500 mg per day and more preferably, the dose is in the range of 300 mg to 400 mg. In an embodiment, the present invention provides a method of treating synucleinopathies in a human subject comprising administering the compound of Formula I or pharmaceutically acceptable salt thereof at a dose selected from 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg or 800 mg per day.

In another aspect the present invention provides a method of treating or preventing synucleinopathies other than Parkinson's disease in a human subject comprising administering compound of Formula 1,

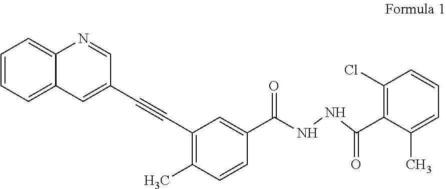 [SMILES: Cc1ccc(C(=O)NNC(=O)c2c(C)cccc2Cl)cc1C#Cc1cnc2ccccc2c1]

or its pharmaceutically acceptable salt thereof at a dose ranging from 5 mg to 500 mg per day.

In another embodiment, the present invention provides a method of treating or preventing synucleinopathies in a human subject comprising administering a compound of Formula I or its pharmaceutically acceptable salt, wherein synucleinopathy is Dementia with Lewy Bodies.

In another embodiment, the present invention provides a method of treating or preventing synucleinopathies in a human subject comprising administering a compound of Formula I or its pharmaceutically acceptable salt, wherein synucleinopathy is multiple system atrophy.

In another embodiment, the present invention provides a method of treating or preventing synucleinopathies in a human subject comprising administering a compound of Formula I or its pharmaceutically acceptable salt, wherein synucleinopathy is associated with REM sleep behavior disorder.

In another embodiment, the present invention provides a method of treating or preventing synucleinopathies in a human subject comprising administering a compound of Formula I or its pharmaceutically acceptable salt, wherein synucleinopathy is associated with neuroautonomic dystrophies and primary autonomic failure.

Suitable pharmaceutically acceptable salts of the compound of the invention may be salts of inorganic acids such as hydrochloric acid, hydrobromic acid, phosphoric acid, and the like or of organic acids such as, for example, acetic acid, benzenesulfonic acid, methanesulfonic acid, benzoic acid, citric acid, glycolic acid, lactic acid, fumaric acid, succinic acid, adipic acid, pimelic acid, suberic acid, azelaic acid, malic acid, tartartic acid, or amino acids, such as glutamic acid or aspartic acid, and the like. One or more hydrogen atoms of the compound of Formula I may be deuteriated i.e. substituted with a deuterium atom.

WIPO publication WO2012098416 (the '416 publication) discloses a markush group of compounds active as c-Abl kinase inhibitors and their usefulness for the treatment of cancers like chronic myelogenous leukemia (CML). Compounds of Formula I of the present invention may be prepared by the processes described in WO2012098416.

The compound of Formula I can be administered orally in the form of a suitable dosage form. A suitable dosage form may include tablet, pellets, capsule, sachet, pellets in sachet, pellets in capsule, powder, granules and the like. The compound of Formula I may be formulated in oral dosage form which may include pharmaceutically acceptable excipients which are in common knowledge of a person skilled in the art. Remington's Pharmaceutical Sciences, Sixteenth Edition, E. W. Martin (Mack Publishing Co., Easton, Pa., 1980) discloses pharmaceutically acceptable carriers which can be used for preparation of a suitable dosage form.

WIPO Publication WO2017208267A1 discloses method of use of compound of Formula I for the treatment of Parkinson's disease. The present invention relates to the use of the compound of Formula I for diseases, other than PD, that are caused by accumulation of alpha-synuclein (aSYN) like Dementia with Lewy Bodies, Pure autonomic failure, REM sleep behavior disorder, Incidental Lewy body disease, Inherited Lewy body diseases, Lewy body dysphagia, and multiple system atrophy. The phrase “compound of Formula I” is used interchangeably with the phrase “compound I” in the present specification and both the phrases refer to a compound having a following structure:

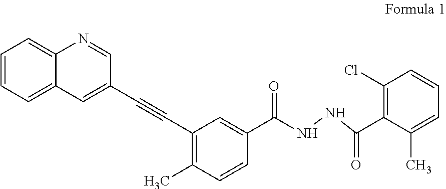 [SMILES: Cc1ccc(C(=O)NNC(=O)c2c(C)cccc2Cl)cc1C#Cc1cnc2ccccc2c1]

The following examples serve to illustrate the invention without limiting the invention in its scope.

### Biological Studies

Neuroprotective Potential of Compound I in a Rat Model of Synucleinopathy

Compound I was evaluated in a adeno-associated virus (AAV) AAV1/2 alpha synuclein rat model based upon AAV1/2-mediated delivery and over-expression of human A53T alpha-synuclein (hA53T-aSYN) in the striatonigral region of the midbrain of female Sprague Dawley (SD) rats (Koprich et al, PLoS One. 7; 6(3):e17698, 2011). The study was designed to assess the ability of Compound I to protect dopaminergic neurons from viral vector mediated over-expression of aSYN leading to neurodegeneration in rat model. This model is typically used as a model for aSYN-induced Parkinsonism, but may serve as a model for aSYN-induced neuronal loss in general. AAV1/2 hA53T-aSYN (a viral vector delivering the capability to express human A53T mutant aSYN), or the control AAV1/2 empty vector, was injected stereotactically and unilaterally, into the striatal region of the midbrain. Rats were treated orally with Compound I (melt extrusion suspension), once daily, to provide equivalent doses of Compound I at 15, 30 and 45 mg/kg.

AAV1/2 hA53T-aSYN or empty AAV1/2 vector was administered sterotactically and unilaterally into the right striatal region on Day 1. Starting on Day 2 and continuing daily until Day 42, animals were fasted for 6 hr prior to oral administration of Compound I or vehicle (placebo). Food was reintroduced 60 minutes later. A total of 5 groups of animals, each group N=12, were employed.

| Group | Initiation | Treatment | N |
|---|---|---|---|
| A | Empty Vector AAV1/2 | Vehicle | 12 |
| B | AAV1/2-hA53T-aSYN | Vehicle | 12 |
| C | | Compound I (15 mg/kg) | 12 |
| D | | Compound I (30 mg/kg) | 12 |
| E | | Compound I (45 mg/kg) | 12 |

Animals were killed for post-mortem assessments on Day 43, at least 18 h after last administration of Compound I or vehicle. Brains were removed along with the terminal blood collection.

As a consequence of localized synucleinopathy caused by the AAV1/2-encoded hA53T aSYN at or near the site of injection, tyrosine hydroxylase (TH)-expressing dopaminergic neurons in the affected striatonigral area degenerate (Koprich et al., PLoS One, 7; 6(3):e17698, 2011). Number of tyrosine hydroxylase positive (TH^{+ve}) cells within the striatonigral region of the right side of the brain (injected side), were assessed using immunohistochemistry and stereological cell counting. Tyrosine hydroxylase is a critical enzyme involved in dopamine biosynthesis and thus, its presence can be used as a marker of live neurons capable of producing dopamine.

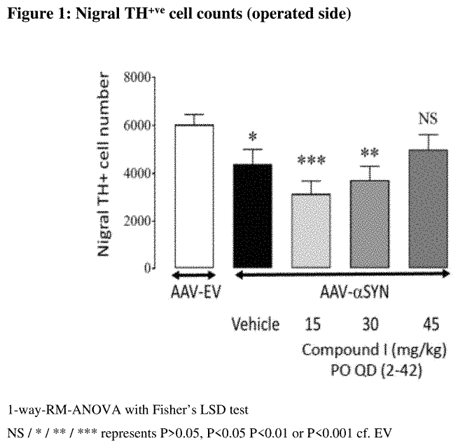

As shown in FIG. 1, animals injected with AAV1/2 encoding hA53T aSYN on the right side of the brain exhibited significant loss (p<0.05) of TH^{+ve} neurons in the striatonigral region compared to the animals that were injected, also on the right side of the brain, with the empty vector AAV1/2 incapable of expressing aSYN and also received vehicle for the treatment period. In contrast, the loss of TH+ve neurons due to synucleinopathy in animals that were injected on the right side of the brain with AAV1/2 encoding hA53T aSYN and treated with different daily oral doses of Compound I was proportional to the dose of Compound I. Treatment with lower doses of Compound I (15 and 30 mg/kg) showed significant reduction (p<0.05) in TH^{+ve} neurons in the striatonigral region compared to the control animals that received empty AAV1/2 incapable of causing synucleinopathy. In contrast, animals injected with AAV1/2 encoding hA53T aSYN and dosed with 45 mg/kg of Compound I did not show significant reduction (p>0.05) in TH^{+ve} neurons in the striatonigral region indicative of the neuroprotective effect of Compound I at this dose.

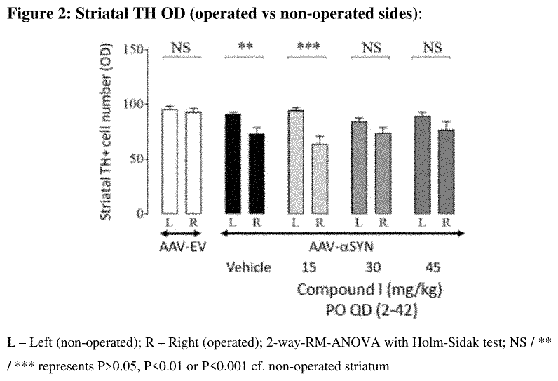

Optical densities of striatonigral TH-expressing neurons from the operated side of the brain of animals, that received the injection of AAV1/2 encoding hA53T aSYN, was compared with that from the non-operated side of the same animals that did not show synucleinopathy. Hence for any treatment, a comparison between the operated side and non-operated side provides a clear reflection of effects on the disease state. As shown in FIG. 2, in animals that received AAV1/2 (empty vector), there was no significant difference in TH^{+ve} optical densities from the left (non-operated) and right (operated) side of the brain. In contrast, animals that received AAV1/2 hA53T aSYN vector on the ride side of the brain (operated side) showed significantly lower (p<0.01) TH^{+ve} optical densities compared to that from the non-operated parts of their brain. Compound I at doses of 30 and 45 mg/kg, but not at 15 mg/kg, showed significant neuroprotective effect reflected in the statistically insignificant difference (p>0.05) between the optical densities from left (non-operated) and right (operated) sides of the brain.

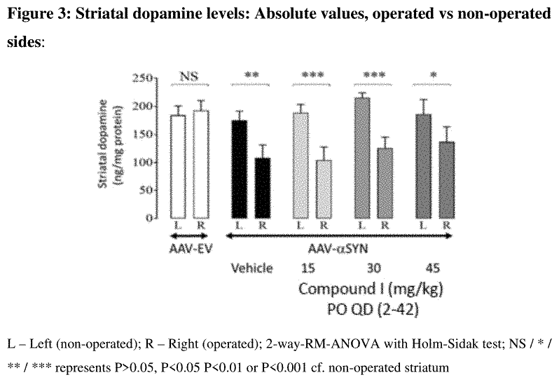

In the example of AAV1/2 hA53T aSYN-induced synucleinopathy studied here, striatonigral TH^{+ve} neurons capable of synthesizing dopamine were shown to be degenerated. Whether or not the neuroprotection of TH^{+ve} dopaminergic neurons conferred by Compound I resulted in the increased production of dopamine was further examined. Hence the total dopamine levels of both the operated right side and non-operated left side of the brain of the animals under study were quantified. As shown in FIG. 3, dopamine levels from the operated diseased right side of the brain were always lower, as a consequence of synucleinopathy-associated neurodegeneration, than their respective non-operated counterparts on the left side.

Compound I at doses 15 and 30 mg/kg failed to restore the dopamine biosynthetic capability in the operated diseased right side of the brain. In contrast, in animals dosed at 45 mg/kg of Compound I, while the dopamine levels produced from the right diseased side of the brain were still lower than that from its left counterpart, there appeared to be a gradual increase in the dopamine producing capability of the diseased part of the brain proportional to the dose of Compound I reflecting its ability to not only protect neurons from degeneration but also help restore their functionality. Administration of Compound I at doses higher than 45 mg/kg may reduce this difference even further.

In conclusion, these studies collectively suggest that Compound I confers significant protection against neurodegeneration caused by synucleinopathy and help restore their functionality. This neuroprotective activity of Compound I supports therapeutic applications of compound I in various disease indications attributed to synucleinopathy.

1. A method of treating or preventing synucleinopathies in a human subject comprising administering a therapeutically effective amount of a compound of Formula 1, 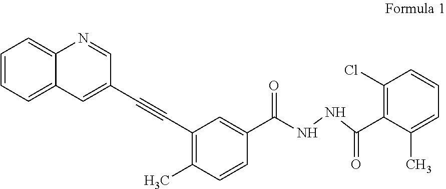 [SMILES: Cc1ccc(C(=O)NNC(=O)c2c(C)cccc2Cl)cc1C#Cc1cnc2ccccc2c1]
or its pharmaceutically acceptable salt thereof.

2. A method of treating or preventing synucleinopathies in a human subject comprising administering compound of Formula 1, 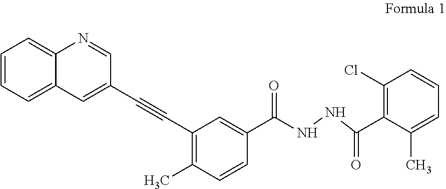 [SMILES: Cc1ccc(C(=O)NNC(=O)c2c(C)cccc2Cl)cc1C#Cc1cnc2ccccc2c1]
or its pharmaceutically acceptable salt at a dose ranging from 0.1 mg to 1000 mg per day.

3. The method of treating or preventing synucleinopathies as in claim 1 wherein, synucleinopathy is Dementia with Lewy Bodies (DLB) or Multiple System Atrophy (MSA) or associated with REM sleep behavior disorder.

4. The method of treating or preventing synucleinopathies as in claim 3 wherein, the compound of Formula I is administered at a dose in the range of 100 mg to 600 mg per day.

5. The method of treating or preventing synucleinopathies as in claim 4 wherein, the compound of Formula I is administered at a dose in the range of 300 mg to 500 mg per day.

6. The method of treating or preventing synucleinopathies as in claim 2 wherein, synucleinopathy is Dementia with Lewy Bodies (DLB) or Multiple System Atrophy (MSA) or associated with REM sleep behavior disorder.

7. The method of treating or preventing synucleinopathies as in claim 6 wherein, the compound of Formula I is administered at a dose in the range of 100 mg to 600 mg per day.

8. The method of treating or preventing synucleinopathies as in claim 7 wherein, the compound of Formula I is administered at a dose in the range of 300 mg to 500 mg per day.
